(19) United States
(12) Patent Application Publication
(10) Pub. No.: US 2011/0130430 A1
(43) Pub. Date: Jun. 2, 2011

(54) Materials Having Embedded Insecticides And Additives

(75) Inventors: Rainer Sonneck, Leverkusen (DE); Thomas Böcker, Leichlinge (DE); Karin Horn, Solingen (DE); Guenther Nentwig, Leverkusen (DE); Maren Heinemann, Bergisch Gladbach (DE); Thomas König, Leverkusen (DE)

(73) Assignee: Bayer CropScience AG, Monheim (DE)

(21) Appl. No.: 12/936,144

(22) PCT Filed: Apr. 1, 2009
(86) PCT No.: PCT/EP2009/002372

§ 371 Date: Dec. 20, 2010

(30) Foreign Application Priority Data
Apr. 4, 2008 (EP) 08154064.3
Jun. 16, 2008 (EP) 08158298.3

(51) Int. Cl. A01N 43/78; A01N 37/34; A01N 53/06; A01N 47/22; A01N 43/40; C07C 59/235; C07C 57/02; A01P 7/04; D01F 1/02

(52) U.S. Cl. 514/365; 514/521; 514/531; 514/480; 514/341; 554/121; 554/1; 264/176.1

(57) ABSTRACT

The present invention concerns polymeric material containing at least an embedded insecticidally active ingredient and an additive, which are released at room temperature. It similarly concerns materials produced from this polymer, for example in the form of self-supporting film/sheet, threads, wovens, fabrics, textiles, nets, curtains and pellets. The invention further concerns processes for producing such polymeric material and also the use of the self-supporting film/sheet, threads, wovens, fabrics, textiles and nets and curtains produced from the material for protecting humans, animals and plants and buildings, machines and packaging against arthropods, particularly for controlling insects.

The present invention concerns polymeric material containing at least an embedded insecticidally active ingredient and an additive, which are released at room temperature. It similarly concerns materials produced from this polymer, for example in the form of self-supporting film/sheet, threads, wovens, fabrics, textiles, nets, curtains and pellets. The invention further concerns processes for producing such polymeric material and also the use of the self-supporting film/sheet, threads, wovens, pellets, fabrics, textiles and nets and curtains produced from the material for protecting humans, animals and plants and buildings, machines and packaging against arthropods, particularly for controlling insects.

It is well known that humans can be protected in their sleep from arthropod stings by insecticidally coated sleeping nets. This is particularly important in countries in which arthropods transmit diseases (malaria for example). Coated wovens can also be used as curtains in front of windows or doors in order to control arthropods entering dwellings. Similarly, using coated wovens to cover vegetable or fruits is known as a way of protecting against arthropods. This makes it possible to minimize insecticide contamination of the plant parts which were later eaten.

Coated materials are efficacious in principle, but have a number of disadvantages. Especially washing the material causes a relatively rapid destruction of the coating, and so a distinct decrease in efficaciousness is observed after just a few wash cycles. This effect has to be counteracted via a high initial loading and/or the addition of binders. In the former case, the surface concentration of insecticidally active ingredient is initially high, which is undesirable from the toxicological viewpoint. The addition of binders is unsatisfactory in that they too are lost by washing, limiting their positive effect on the washfastness of coated materials.

The known materials for nets are essentially polyester and polyethylene, which have limited durability (polyester in particular) and in some instances are perceived as surfaces which are unpleasantly brittle to the touch (polyethylene in particular). Therefore, it would be desirable to develop materials based on other, more durable, mechanically stronger polymers.

In crop protection, additives have already been used very successfully for years to reduce the use of active ingredients. Additives in this connection are substances which themselves have no insecticidal effect, but enhance the insecticidal effect of simultaneously applied actives. This is accomplished, for example, by improving the penetration of the active ingredient through the plant or arthropod cuticle or by inhibiting the metabolization of the active ingredient in the target organism/plant. Owing to the effect of the additives, it is possible to reduce the use of active ingredients, which reduces the exposure of users and consumers and also improves environmental compatibility.

EP 1 648 230 discloses a process for producing pyrethroid-containing polymer for use in nets. The active ingredient is not used directly, but in the form of a covalent associate with a second substance, which must have a C—C double bond. This associate is then initially processed with a polymer to form a highly concentrated intermediate product (masterbatch) which is then in turn processed to the end product. This process is inconvenient, and so there is a need for simplification. EP 1 648 230 states that a reaction occurs in the course of the process between the double bond of the chrysanthemate radical in the pyrethroid and the second substance. As a result, the process is specific for pyrethroids or at least for such insecticidal actives as have a C—C double bond of similar reactivity. Chemical conversion of the insecticidal active is also problematical because it is likely to involve a reduction in or even a complete loss of efficacy.

A net containing an active ingredient from the class of the pyrethroids and an additive (piperonyl butoxide) is disclosed in ZA 200509810. However, there is no disclosure there as to how the polymers are produced, to what extent the nets are actually insecticidally efficacious, how long they remain efficacious or, more particularly, to what extent the presence of the piperonyl butoxide is actually advantageous. More particularly, there is no disclosure or suggestion as to what extent other additives can be successfully used in polymer-based materials. A masterbatch process is mentioned but in no way directly described.

The use of polypropylene is also known from insecticidal evaporator platelets (for example WO 97/29634, WO 99/01030, WO 05/044001). In insecticidal evaporator platelets, an insecticidally active ingredient is embedded into a polypropylene matrix and quickly released by heating to above 100° C. in order to treat the room for example. A room-temperature use or the use in long-acting materials is not described there, nor a combination with additives.

It is an object of the present invention to provide novel materials that achieve at least one of the following objects:
good insecticidal effect
reducing the concentration of active ingredient while maintaining insecticidal efficacy
fast-acting insecticidal efficacy
long-lasting insecticidal efficacy
uniform release of active ingredient
long durability
simple production

We have found that these objects are achieved by the polymers of the present invention, which are selected from polyethylene and polypropylene and incorporate
a) at least one insecticidally active ingredient selected from organophosphates, pyrethroids, neonicotinoids and carbamates,
b) at least one additive selected from sebacic esters, fatty acids, fatty acid esters, vegetable oils, esters of vegetable oils, alcohol alkoxylates and antioxidants.

It is surprising that the combination of active and additive displays a synergistic effect from the matrix of the present invention, since this synergistic effect had hitherto only been demonstrated for use in solution. Since, however, the matrix of the present invention is a solid polymeric material, a person skilled in the art would not consider it obvious to transfer this principle. This is because a synergism requires cooperation by the two components, ie, they have to be present relative to each other in a ratio that is characteristic for the chemical entities in question. As a result, the two components have to have matching diffusion dynamics in order that they may be present at all times on the surface of the material in the characteristic ratio relative to each other. Since daily use causes removal of the chemical entities from the surface, each individual chemical entity has to be replenished in such a suitable way that the characteristic ratio responsible for the synergistic effect is reestablished. In a liquid, in which the components are free to move and the distribution of the components is homogeneous, this is much simpler than in a solid phase, in which the two components may exhibit completely different diffusion characteristics, for example accumulation at the surface. In addition, production of the materials of the present invention involves exposure to thermal stresses which far exceed those involved in the production of liquid formulations. The influence of these extreme conditions was likewise impossible to judge by a person skilled in the art, particularly since some of the additives and insecticides contain fundamentally reactive or thermolabile groups such as double bonds and ester groupings for example.

Active ingredients which can be used according to the present invention are those from the classes of the organophosphates, pyrethroids, neonicotinoids and carbamates.

Organophosphates include for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos(-methyl/-ethyl), coumaphos, cyanofenphos, cyanophos, chlorfenvinphos, demeton-5-methyl, demeton-5-methylsulphon, dialifosi, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion.

The pyrethroids include for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-Cypermethrin, cis-Resmethrin, cis-Permethrin, clocythrin, cycloprothrin, cyfluthrin, cyhalothrin, cypermethrin (alpha-, beta-, theta-, zeta-), cyphenothrin, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-Fluvalinate, tefluthrin, terallethrin, tetramethrin (−1R— isomer), tralomethrin, transfluthrin, ZXI 8901 and pyrethrin (pyrethrum). Preference according to the present invention is given to beta-cyfluthrin, bifenthrin, cyfluthrin, deltamethrin and transfluthrin. Particular preference according to the present invention is given to cyfluthrin, deltamethrin and transfluthrin.

The neonicotinoids include for example acetamiprid, clothianidin, dinotefuran, imidacloprid, nitenpyram, nithiazine, thiacloprid and thiamethoxam. Preference according to the present invention is given to imidacloprid and clothianidin.

The carbamates include for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulfan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb and triazamate. Preference according to the present invention is given to bendiocarb and carbaryl.

The insecticidally active ingredient a) likewise comprises mixtures between the active ingredients mentioned.

Additives b) according to the present invention are for example sebacic esters, fatty acids, fatty acid esters, vegetable oils, esters of vegetable oils, alcohol alkoxylates and antioxidants.

Suitable sebacic esters are for example dimethyl sebacate, diethyl sebacate, dibutyl sebacate, dibenzyl sebacate, bis(N-succinimidyl)sebacate, bis(2-ethylhexyl)sebacate, bis(1-octyloxy-2,2,6,6-tetramethyl-4-piperidyl)sebacate, bis(2,2,6,6-tetramethyl-4-piperidyl)sebacate and bis(1,2,2,6,6-pentamethyl-4-piperidinyl)sebacate (BLS292).

Suitable fatty acids are (preferably mono- or polyunsaturated) fatty acids having a chain length of 12 to 24 carbon atoms, for example palmitoleic acid, oleic acid, elaidic acid, vaccenic acid, icosenic acid, cetoleic acid, erucic acid, nervonic acid, linoleic acid, alpha-linolenic acid, gamma-linolenic acid, arachidonic acid, timnodonic acid, clupanodonic acid and cervonic acid. Particular preference is given to oleic acid, linoleic acid, alpha-linolenic acid and gamma-linolenic acid.

Suitable fatty acid esters are preferably methyl or ethyl esters of the above-recited fatty acids. Methyl esters are particularly preferred.

Fatty acids and their esters can each also be present in mixtures.

Useful vegetable oils include all plant-derivable oils customarily usable in agrochemical compositions. As examples there may be mentioned sunflower oil, rapeseed oil, olive oil, castor oil, colza oil, maize kernel oil, cottonseed oil and soybean oil. Rapeseed oil is preferred.

Suitable esters of vegetable oils are methyl or ethyl esters of the above-recited oils. Methyl esters are preferred.

Alcohol alkoxylates according to the present invention are those of formula (I)

R—O-(EO)ₘ—R′ (I)

where
R represents branched or unbranched C₈-C₁₅-alkyl,
m represents 5 to 15,
R′ represents hydrogen or C₁-C₆-alkyl, and
E represents CH₂—CH₂.

Preference is given to alcohol alkoxylates in which R represents a branched C₁₂-C₁₄-alkyl, m represents 6 to 10 and R′ represents hydrogen. Such alcohol alkoxylates are commercially available (Lutensol® range, BASF).

Alcohol alkoxylates are produced in a polymerization process and so are present as mixtures of homologous substances differing in chain length m, so that m can also represent non-integral average values.

Antioxidants useful as additives include for example butylhydroxytoluene, butylhydroxyanisole and L-ascorbic acid.

The combinations recited in the table below represent preferred combinations of active and additive. In effect, each of the combinations mentioned is a preferred combination.

TABLE 1
combinations of active and additive

| | | |
|---|---|---|
| 1 | cyfluthrin | dimethyl sebacate |
| 2 | cyfluthrin | diethyl sebacate |
| 3 | cyfluthrin | dibutyl sebacate |
| 4 | cyfluthrin | dibenzyl sebacate |
| 5 | cyfluthrin | bis(N-succinimidyl) sebacate |
| 6 | cyfluthrin | bis(2-ethylhexyl) sebacate |
| 7 | cyfluthrin | bis(1-octyloxy-2,2,6,6-tetramethyl-4-piperidyl) |
| | | sebacate |
| 8 | cyfluthrin | bis(2,2,6,6-tetramethyl-4-piperidyl) sebacate |
| 9 | cyfluthrin | bis(1,2,2,6,6-pentamethyl-4-piperidinyl) sebacate |
| | | (BLS292) |
| 10 | cyfluthrin | oleic acid |
| 11 | cyfluthrin | linoleic acid |
| 12 | cyfluthrin | alpha-linolenic acid |
| 13 | cyfluthrin | gamma-linolenic acid |
| 14 | cyfluthrin | methyl ester |
| 15 | cyfluthrin | rapeseed oil |
| 16 | cyfluthrin | alcohol alkoxylates |
| 17 | cyfluthrin | butylhydroxytoluene |
| 18 | deltamethrin | dimethyl sebacate |
| 19 | deltamethrin | diethyl sebacate |
| 20 | deltamethrin | dibutyl sebacate |
| 21 | deltamethrin | dibenzyl sebacate |
| 22 | deltamethrin | bis(N-succinimidyl) sebacate |
| 23 | deltamethrin | bis(2-ethylhexyl) sebacate |
| 24 | deltamethrin | bis(1-octyloxy-2,2,6,6-tetramethyl-4-piperidyl) |
| | | sebacate |
| 25 | deltamethrin | bis(2,2,6,6-tetramethyl-4-piperidyl) sebacate |
| 26 | deltamethrin | bis(1,2,2,6,6-pentamethyl-4-piperidinyl) sebacate |
| | | (BLS292) |
| 27 | deltamethrin | oleic acid |
| 28 | deltamethrin | linoleic acid |
| 29 | deltamethrin | alpha-linolenic acid |
| 30 | deltamethrin | gamma-linolenic acid |
| 31 | deltamethrin | methyl ester |
| 32 | deltamethrin | rapeseed oil |
| 33 | deltamethrin | alcohol alkoxylates |
| 34 | deltamethrin | butylhydroxytoluene |
| 35 | transfluthrin | dimethyl sebacate |
| 36 | transfluthrin | diethyl sebacate |
| 37 | transfluthrin | dibutyl sebacate |
| 38 | transfluthrin | dibenzyl sebacate |
| 39 | transfluthrin | bis(N-succinimidyl) sebacate |
| 40 | transfluthrin | bis(2-ethylhexyl) sebacate |
| 41 | transfluthrin | bis(1-octyloxy-2,2,6,6-tetramethyl-4-piperidyl) |
| | | sebacate |
| 42 | transfluthrin | bis(2,2,6,6-tetramethyl-4-piperidyl) sebacate |
| 43 | transfluthrin | bis(1,2,2,6,6-pentamethyl-4-piperidinyl) sebacate |
| | | (BLS292) |
| 44 | transfluthrin | oleic acid |
| 45 | transfluthrin | linoleic acid |
| 46 | transfluthrin | alpha-linolenic acid |
| 47 | transfluthrin | gamma-linolenic acid |
| 48 | transfluthrin | methyl ester |
| 49 | transfluthrin | rapeseed oil |
| 50 | transfluthrin | alcohol alkoxylates |
| 51 | transfluthrin | butylhydroxytoluene |
| 52 | imadacloprid | dimethyl sebacate |
| 53 | imadacloprid | diethyl sebacate |
| 54 | imadacloprid | dibutyl sebacate |
| 55 | imadacloprid | dibenzyl sebacate |
| 56 | imadacloprid | bis(N-succinimidyl) sebacate |
| 57 | imadacloprid | bis(2-ethylhexyl) sebacate |
| 58 | imadacloprid | bis(1-octyloxy-2,2,6,6-tetramethyl-4-piperidyl) |
| | | sebacate |
| 59 | imadacloprid | bis(2,2,6,6-tetramethyl-4-piperidyl) sebacate |
| 60 | imadacloprid | bis(1,2,2,6,6-pentamethyl-4-piperidinyl) sebacate |
| | | (BLS292) |
| 61 | imadacloprid | oleic acid |
| 62 | imadacloprid | linoleic acid |
| 63 | imadacloprid | alpha-linolenic acid |
| 64 | imadacloprid | gamma-linolenic acid |
| 65 | imadacloprid | methyl ester |
| 66 | imadacloprid | rapeseed oil |
| 67 | imadacloprid | alcohol alkoxylates |
| 68 | imadacloprid | butylhydroxytoluene |
| 69 | clothianidin | dimethyl sebacate |
| 70 | clothianidin | diethyl sebacate |
| 71 | clothianidin | dibutyl sebacate |
| 72 | clothianidin | dibenzyl sebacate |
| 73 | clothianidin | bis(N-succinimidyl) sebacate |
| 74 | clothianidin | bis(2-ethylhexyl) sebacate |
| 75 | clothianidin | bis(1-octyloxy-2,2,6,6-tetramethyl-4-piperidyl) |
| | | sebacate |
| 76 | clothianidin | bis(2,2,6,6-tetramethyl-4-piperidyl) sebacate |
| 77 | clothianidin | bis(1,2,2,6,6-pentamethyl-4-piperidinyl) sebacate |
| | | (BLS292) |
| 78 | clothianidin | oleic acid |
| 79 | clothianidin | linoleic acid |
| 80 | clothianidin | alpha-linolenic acid |
| 81 | clothianidin | gamma-linolenic acid |
| 82 | clothianidin | methyl ester |
| 83 | clothianidin | rapeseed oil |
| 84 | clothianidin | alcohol alkoxylates |
| 85 | clothianidin | butylhydroxytoluene |
| 86 | bendiocarb | dimethyl sebacate |
| 87 | bendiocarb | diethyl sebacate |
| 88 | bendiocarb | dibutyl sebacate |
| 89 | bendiocarb | dibenzyl sebacate |
| 90 | bendiocarb | bis(N-succinimidyl) sebacate |
| 91 | bendiocarb | bis(2-ethylhexyl) sebacate |
| 92 | bendiocarb | bis(1-octyloxy-2,2,6,6-tetramethyl-4-piperidyl) |
| | | sebacate |
| 93 | bendiocarb | bis(2,2,6,6-tetramethyl-4-piperidyl) sebacate |
| 94 | bendiocarb | bis(1,2,2,6,6-pentamethyl-4-piperidinyl) sebacate |
| | | (BLS292) |
| 95 | bendiocarb | oleic acid |
| 96 | bendiocarb | linoleic acid |
| 97 | bendiocarb | alpha-linolenic acid |
| 98 | bendiocarb | gamma-linolenic acid |
| 99 | bendiocarb | methyl ester |
| 100 | bendiocarb | rapeseed oil |
| 101 | bendiocarb | alcohol alkoxylates |
| 102 | bendiocarb | butylhydroxytoluene |
| 103 | carbaryl | dimethyl sebacate |
| 104 | carbaryl | diethyl sebacate |
| 105 | carbaryl | dibutyl sebacate |
| 106 | carbaryl | dibenzyl sebacate |
| 107 | carbaryl | bis(N-succinimidyl) sebacate |
| 108 | carbaryl | bis(2-ethylhexyl) sebacate |
| 109 | carbaryl | bis(1-octyloxy-2,2,6,6-tetramethyl-4-piperidyl) |
| | | sebacate |
| 110 | carbaryl | bis(2,2,6,6-tetramethyl-4-piperidyl) sebacate |
| 111 | carbaryl | bis(1,2,2,6,6-pentamethyl-4-piperidinyl) sebacate |
| | | (BLS292) |
| 112 | carbaryl | oleic acid |
| 113 | carbaryl | linoleic acid |
| 114 | carbaryl | alpha-linolenic acid |
| 115 | carbaryl | gamma-linolenic acid |
| 116 | carbaryl | methyl ester |
| 117 | carbaryl | rapeseed oil |
| 118 | carbaryl | alcohol alkoxylates |
| 119 | carbaryl | butylhydroxytoluene |

The concentration of the insecticidally active ingredient in the polymeric material can be varied within a relatively wide concentration range (for example from 0.01% to 2% by weight). The concentration should be chosen according to the field of application such that the requirements concerning insecticidal efficacy, durability and toxicity are met. Adapting the properties of the material can also be accomplished by mixing insecticides in the polymeric material, by the blending of materials according to the present invention which contain different insecticides, or by using materials according to the present invention which contain different insecticides and which are used in combination with each other, for example as mosaic nets. Custom-tailored wovens are obtainable in this way.

The concentration of the additive in the polymer can likewise be varied within a relatively wide concentration range. The concentration should be chosen such that a very pronounced synergism with the insecticide present may occur over a very long period.

Suitable selection of the combination of insecticide and additive at incorporation in polyethylene or polypropylene provides sufficient efficacy against animal pests on the surface as long as sufficient bioavailable active is present on the surface. The delivery rate of the composition of the present invention on the surface of polyethylene or polypropylene nets is chosen such that full efficacy is retained for 60 washes.

The polymeric material of the present invention can be further processed into miscellaneous products by processes adapted to the base material. These products include for example foils, pellets, plates, air-cushioning materials, films, profiles, sheets, wires, threads, tapes, cable and pipe linings, casings for electrical instruments (for example in switch boxes, aircraft, refrigerators, etc.).

The materials of the present invention and threads, wovens, nets, etc. produced therefrom are very useful for killing harmful or annoying arthropods, more particularly arachnids and insects.

Arachnids include mites (e.g. Sarcoptes scabiei, Dermatophagoides pteronys-sinus, Dermatophagoides farinae, Dermanyssus gallinae, Acarus siro) and ticks (e.g. Ixodes ricinus, Ixodes scapularis, Argas reflexus, Ornithodorus moubata, Boophilius microplus, Amblyomma hebraeum, Rhipicephalus sanguineus).

Sucking insects include essentially the mosquitoes (e.g. Aedes aegypti, Aedes vexans, Culex quinquefasciatus, Culex tarsalis, Anopheles albimanus, Anopheles stephensi, Mansonia titillans), sand flies (e.g. Phlebotomus papatasii), gnats (e.g. Culicoides furens), black flies (e.g. Simulium damnosum), biting houseflies (e.g. Sto-moxys calcitrans), Tsetse flies (e.g. Glossina morsitans morsitans), horseflies (e.g. Taba-nus nigrovittatus, Haematopota pluvialis, Chrysops caecutiens), common houseflies (e.g. Musca domestica, Musca autumnalis, Musca vetustissima, Fannia canicularis), flesh flies (e.g. Sarcophaga carnaria), myiasis-causing flies (e.g. Lucilia cuprina, Chrysomyia chloro-pyga, Hypoderma bovis, Hypoderma lineatum, Dermatobia hominis, Oestrus ovis, Gaste-rophilus intestinalis, Cochliomyia hominivorax), bugs (e.g. Cimex lectularius, Rhodnius prolixus, Triatoma infestans), lice (e.g. Pediculus humanis, Haematopinus suis, Damalina ovis), fleas (e.g. Pulex irritans, Xenopsylla cheopis, Ctenocephalides canis, Ctenocephali-des felis) and sand fleas (Tunga penetrans).

Biting insects include essentially cockroaches (e.g. Blattella germanica, Periplaneta americana, Blatta orientalis, Supella longipalpa), beetles (e.g. Sitiophilus granarius, Tenebrio molitor, Dermestes lardarius, Stegobium paniceum, Anobium punctatum, Hylotrupes bajulus), termites (e.g. Reticulitermes lucifugus), ants (e.g. Lasius niger, Monomorium pharaonis), wasps (e.g. Vespula germanica) and larvae of moths (e.g. Ephestia elutella, Ephestia cautella, Plodia interpunctella, Hofmannophila pseudospretella, Tineola bisselliella, Tinea pellionella, Trichophaga tapetzella).

The materials of the present invention are preferably used against insects, particularly of the order Diptera and more preferably against the suborder Nematocera.

In addition to at least one active ingredient from the classes of the organophosphates, pyrethroids, carbamates or neonicotinoids, the polymer according to the invention may contain one or more further insecticidally active ingredients. Suitable are for example DDT, indoxacarb, nicotine, bensultap, cartap, spinosad, camphechlor, chlordane, endosulfan, gamma-HCH, HCH, heptachlor, lindane, methoxychlor, acetoprole, ethiprole, fipronil, pyrafluprole, pyriprole, vaniliprole, avermectin, emamectin, emamectin-benzoate, ivermectin, milbemycin, diofenolan, epofenonane, fenoxycarb, hydroprene, kinoprene, methoprene, pyriproxifen, triprene, chromafenozide, halofenozide, methoxyfenozide, tebufenozide, bistrifluoron, chlofluazuron, diflubenzuron, fluazuron, flucycloxuron, flufenoxuron, hexaflumuron, lufenuron, novaluron, noviflumuron, penfluoron, teflubenzuron, triflumuron, buprofezin, cyromazine, diafenthiuron, azocyclotin, cyhexatin, fenbutatin-oxide, chlorfenapyr, binapacyrl, dinobuton, dinocap, DNOC, fenazaquin, fenpyroximate, pyrimidifen, pyridaben, tebufenpyrad, tolfenpyrad, hydramethylnon, dicofol, rotenone, acequinocyl, fluacrypyrim, Bacillus thuringiensis strains, spirodiclofen, spiromesifen, spirotetramat, 3-(2,5-dimethylphenyl)-8-methoxy-2-oxo-1-azaspiro[4.5]dec-3-en-4-yl ethyl carbonate (alias: carbonic acid, 3-(2,5-dimethylphenyl)-8-methoxy-2-oxo-1-azaspiro[4.5]dec-3-en-4-yl ethyl ester, CAS-Reg.-No.: 382608-10-8), flonicamid, amitraz, propargite, flubendiamide, chloranthraniliprol, thiocyclam hydrogen oxalate, thiosultap-sodium, azadirachtin, Bacillus spec., Beauveria spec., Codlemone, Metarrhizium spec., Paecilomyces spec., Thuringiensin, Verticillium spec., aluminium phosphid, methylbromide, sulfurylfluorid, cryolite, flonicamid, pymetrozine, clofentezine, etoxazole, hexythiazox, amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonylbutoxid, kaliumoleat, pyridalyl, sulfluramid, tetradifon, tetrasul, triarathene and verbutin.

The self-supporting film/sheet, threads, wovens, pellets, fabrics, textiles, nets and curtains produced from the material of the present invention are used for protecting humans, animals and plants and buildings (for example wall lining for silos and storage facilities), and also building parts (for example roofing membranes, curtain-type facades), machines (airconditionings, electronic and servorooms) and packaging (for example boxes and containers for clothing transport) against arthropods, particularly for controlling insects.

### Producing the Polymers of the Present Invention

The polymeric materials of the present invention are produced by mixing the insecticide and the additive with the polymer in the liquid phase.

For this, the polymer is preferably melted in a first step. Useful apparatus for melting includes for example a single-screw extruder, a twin-screw extruder, a multi-screw extruder or a co-kneader.

Single-screw extruders are described for example in “Der Einschneckenextruder—Grundlagen und Systemoptimierung”, Gerhard A. Martin, VDI-Verlag, ISBN 3-18-234247-9. The single-screw extruder used can be for example a smooth or grooved barrel extruder or a Transfermix. A grooved barrel extruder is preferred.

Twin-screw extruders are described for example in “Der Doppelschneckenextruder—Grundlagen und Beispiele”, VDI-Gesellschaft Kunststofftechnik, ISBN 3-18-234201-0 or in “Der gleichläufige Doppelschneckenextruder”, Klemens Kohlgrüber, Hanser Verlag, ISBN 978-3-446-41251-1. The twin-screw extruder may be either co- or counter-rotating. Twin-screw extruders may further be close-meshing or non-intermeshing. Preference is given to a close-meshing corotating design.

Multi-screw extruders have at least three screws, preferably four to twelve. The screws may each be arranged to form close-meshing pairs, in which case the screw pairs can be arranged tangentially and counter-rotating relative to each other. The screws of a multi-screw extruder can further be all corotating, in which case each screw intermeshes in two neighbouring screws. A special form of multi-screw extruder is the planetary roll extruder wherein a driven central spindle drives freely revolving planetary spindles which in turn circulate in a fixed housing. The central spindle, the planetary spindles and the housings have toothed-wheel intermeshing.

The construction of the extruder screw is adapted to the particular application scenario.

Room temperature solid insecticides are metered together with the starting polymer pellets into the feed zone of the extruder. The extruder housings are temperature-controlled to 200° C. In the extruder, the polymer and depending on its melting point, the insecticide as well are melted and mixed. The mixture is extruded through a hole die and pelletized.

The mixing of the insecticide and of the additive with the molten polymer can take place in the same apparatus in which the melting of the polymer takes place, or in a further apparatus. All the abovementioned extruders are suitable for the mixing. A further possibility is to mix the insecticide and the additive with the polymer in a static mixer. Static mixers are described for example in “Plastverarbeiter”, 11(43), 1992, “Statisches Mischen in der Kunststoffverarbeitung und -herstellung”.

The insecticide and the additive can be added in liquid or solid form. The insecticide can be metered, in both solid and liquid form, together with the solid polymer, through a separate channel into the solids-conveying region, or into the polymer melt. Metered addition of the insecticide or of the additive via two or more points of addition is also possible. This can be sensible particularly when different insecticides or additives are to be mixed into the polymer concurrently.

Preferably, the melting of the polymer and the incorporation of the insecticide and additive take place in one apparatus.

When the insecticide or the additive is added in liquid form, it is generally melted and intermediately stored in an initial charge vessel, from which it is then conveyed into the mixing apparatus. The conveying can be effected for example via a pump or via an increased admission pressure. The temperature of the initial charge vessel is chosen such that the insecticide is stable and the viscosity of the insecticide is sufficiently small to ensure good pumpability. It is advantageous in this case to heat the initial charge vessel, the pump and all lines. The metering into the mixing apparatus preferably proceeds via a needle valve. The metered amount of insecticide is preferably measured by a suitable mass flow rate meter, for example according to the Coriolis principle or according to the heated wire principle, and closed-loop controlled to small deviations via the pump or a valve.

Room temperature liquid insecticides are added to the already molten polymer in a processing zone of the extruder via a needle valve. Depending on the viscosity and melting point of the insecticide, the insecticide is heated for this.

After mixing, a preferred embodiment comprises cooling and solidifying of the polymeric materials and also subdivision into pellets. This can be accomplished for example using the common strand pelletization process wherein one or more dies extrude continuous strands which are then air or water cooled to solidify them and subsequently comminuted to the desired size in a pelletizer. Underwater pelletization is a further method, the melt emerging from the die underwater, being cut there and by a circulating blade and subsequently water cooled, thereafter screened off and dried.

The resulting pellets of the polymeric material of the present invention are then further processed into the applications of the present invention such as, for example, self-supporting film/sheet, threads or tapes (see page 10 lines 21 to 24).

In a preferred embodiment of the invention, it is only polymeric material produced by the mixing operation which is sent to the further-processing operation. The amount of insecticide or additive in the simple mixing operation is in the range from 0.05% to 5% by weight, preferably in the range from 0.5% to 1.5% by weight.

In a further embodiment, a polymeric material having an increased concentration of insecticide or in pellet form is produced (known as a masterbatch) and sent for further processing in a mixture with polymer not mixed with insecticide. In this case, the concentration of insecticide or additive in the polymeric material is increased, preferably to a concentration between 5% to 20% by weight, preferably 8% to 15% by weight.

The residence times in which the polymer is liquid during melting and mixing are between 3 and 300 seconds, more preferably between 5 and 120 seconds and more preferably between 8 and 30 seconds.

In a further preferred embodiment, the polymeric material is sent for further processing immediately after mixing, in the form of a melt. The further-processing operation is preferably a spinning process. In this process, threads are subsequently produced by melt spinning as described for example in DE A 41 36 694 (page 2 lines 27-38, page 5 line 45-page 6 line 23) or DE-A 10 2005 054 653 ([0002]).

### Biological Effect of Polymers of the Present Invention

The examples which follow illustrate the good insecticidal efficacy of the polymeric composition of the present invention. While self-supporting films containing a single active ingredient display infirmities in their efficacy, materials containing an active ingredient and an additive display an efficacy beyond that of a simple addition of efficacies.

Insecticides and additives are said to display a synergistic effect whenever the efficacy of their mixture is greater than the sum total of the efficacies of the individually applied substances.

The expected efficacy of a given combination of two substances can be calculated as follows after S. R. Colby, Weeds 15 (1967), 20-22:

when
X is the kill percentage, expressed in % of the untreated control, using the active ingredient A in a concentration of m g/kg,
Y is the kill percentage, expressed in % of the untreated control, using the additive in a concentration of n g/kg, and
E is the expected kill percentage, expressed in % of the untreated control, on using the active ingredient A and the additive in application rates of m and n g/ha or in a concentration of m and n ppm,
   then

$$E is = X + Y - \frac{X \cdot Y}{100}$$

When the actual insecticidal kill percentage is greater than that calculated, the kill percentage attributable to the combination is superadditive, ie, there is a synergistic effect. In this case, the actually observed kill percentage has to be greater than the expected kill percentage (E) calculated from the above formula.

### Test Methods

### Test Insects

Female malaria mosquitoes (Anopheles gambiae, susceptible Kisumu strain), fed with sugared water only.

### Samples

The polymeric materials were produced using a corotating close-meshing twin-screw extruder. Extruder temperature was 200° C. in all steps and extruder speed was 160 rpm. A first step comprised producing a mixture of 3% by weight of technical-grade deltamethrin and 97% by weight of polypropylene (TK3). The polypropylene used contains the customary additives known for example from WO-A 04/094122 (page 5 line 22 to page 15 line 4). The two materials were introduced in solid form into the feed zone of the extruder. This mixture was diluted in a second step to a polymer material containing 1% by weight of deltamethrin (TK1). To this end, 33.33% by weight of TK3 and 66.67% by weight of polypropylene were mixed in a tumble mixer and this mixture was extruded using a corotating close-meshing twin-screw extruder under the abovementioned conditions.

In the third step, 1% by weight of the additive (oleic acid or rapeseed oil) was mixed into 99% by weight of polypropylene using a corotating close-meshing twin-screw extruder. The polypropylene was supplied to the extruder in pellet form in the feed zone and the additive was metered in liquid form via a needle valve into the polymer melt in a later housing zone. Extrusion took place under the abovementioned conditions.

To produce the polymeric material of the present invention, 10% by weight of the insecticide-containing polymeric material (TK1) were mixed with 25% by weight of the additive-containing polymeric material and 65% by weight of polypropylene in a tumble mixer and this mixture was extruded using a corotating close-meshing twin-screw extruder under the previously mentioned conditions.

The polymeric material of the present invention was used to produce self-supporting films from 25 to 50 μm in thickness. To this end, the polymeric material was melted in a single-screw extruder temperature controlled to 220° C. and extruded through a wide-slot die. The extruded films were hauled off using a polishing stack. The temperature of the first roll of the polishing stack was about 85° C. and the temperature of the second roll of the polishing stack was about 60° C.

### Three-Minute Exposure (Cylinder Test)

The tests were carried out using the “WHO Adult Mosquito Susceptibility Test Kit” with an exposure time of 3 minutes on part-samples. The samples were 12×15 cm in size.

Knock-down was determined after 5, 10, 15, 20, 30, 40, 50 and 60 minutes. Thereafter, the mosquitoes were given water with 5% sugar for 24 hours and then mortality was redetermined. Each test consisted of three rounds, which were averaged.

The KT50 and KT95 values were calculated using the Excel-Add-In XLfit 3.0 (ID Business Solutions Ltd., Guildford, England). The 205 model with set thresholds 0% and 100% was used.

### Washing Operation

To remove any surface residues, the samples were washed once as follows:

500 ml of deionized water containing 0.2% (w/v) of laundry detergent (Le Chat, Henkel, France) were introduced at 30° C. into a 1 liter glass bottle. Three sample pieces 12×15 cm in size were introduced into the bottle which stood on a horizontal shaker (155 movements per minute) in a water bath at 30° C. Thereafter, the water was poured out of the bottle and the sample was rinsed twice with 500 ml of water each time for 10 minutes again under shaking.

The film samples were line dried for two hours and thereafter additionally for at least 24 hours in a line state before being washed again.

### Results

TABLE 2
Knock-down and mortality

| | | | | | | | | | % |
|---|---|---|---|---|---|---|---|---|---|
| Active ingredient | % knock-down after | | | | | | | | mortality |
| (+additive) | 5′ | 10′ | 15′ | 20′ | 30′ | 40′ | 50′ | 60′ | 24 h |
| 0.10% deltamethrin | 0 | 9 | 8 | 9 | 26 | 38 | 49 | 58 | 74 |
| 0.10% deltamethrin + | 0 | 4 | 9 | 13 | 43 | 51 | 57 | 77 | 88 |
| 0.25% oleic acid | | | | | | | | | |
| 0.25% oleic acid | 0 | 0 | 2 | 2 | 2 | 2 | 2 | 2 | 26 |
| 0.10% deltamethrin + | 4 | 2 | 11 | 26 | 40 | 57 | 70 | 81 | 87 |
| 0.25% rapeseed oil | | | | | | | | | |
| (refined) | | | | | | | | | |
| 0.25% rapeseed oil | 2 | 2 | 2 | 2 | 2 | 4 | 4 | 4 | 34 |
| (refined) | | | | | | | | | |

1. A polymer selected from polyethylene and polypropylene and incorporating
a) at least one insecticidally active ingredient selected from organophosphates, pyrethroids, neonicotinoids and carbamates,
b) at least one additive selected from sebacic esters, fatty acids, fatty acid esters, vegetable oils, esters of vegetable oils, alcohol alkoxylates and antioxidants.

2. The polymer according to claim 1, wherein the polymer is propylene and the active ingredient is deltamethrin, cyfluthrin, transfluthrin, bendiocarb, carbaryl, imidacloprid or clothianidine.

3. The polymer according to claim 1, wherein the additive is rapeseed oil or oleic acid.

4. Pellets, self-supporting film/sheet or plates containing polymer according to claim 1.

5. Fibers or threads containing polymer according to claim 1.

6. Wovens containing fibers or threads according to claim 5.

7. A sleeping net, netting, hammock or curtain containing fibers or threads according to claim 5.

8. The process for producing a polymer according to claim 1 by extrusion of a polyethylene or polypropylene starting material with addition of insecticide and additive wherein the addition
in the case of a room temperature solid insecticide takes place together with the starting material into the feed zone of the extruder and
in the case of a room temperature liquid insecticide is effected in a processing zone of the extruder via a needle valve.

9. The use of sebacic esters, fatty acids, fatty acid esters, vegetable oils, esters of vegetable oils, alcohol alkoxylates or antioxidants for improving the efficacy of polyethylene or polypropylene incorporating at least one insecticide selected from organophosphates, pyrethroids, neonicotinoids and carbamates.

10. The use of the pellets, self-supporting film/sheet or plates according to claim 4 and also of the sleeping nets, nettings, hammocks or curtains according to claim 7 for protecting humans, animals, plants, buildings, building parts, machines or packaging against arthropods.
